(19) United States
(12) Patent Application Publication
(10) Pub. No.: US 2016/0199226 A1
(43) Pub. Date: Jul. 14, 2016

(54) FLUID HANDLING AND DISPENSING TOOL

(71) Applicant: Jack Wayne Colgan, Safety Harbor, FL (US)

(72) Inventor: Jack Wayne Colgan, Safety Harbor, FL (US)

(21) Appl. No.: 14/934,391

(22) Filed: Nov. 6, 2015

Related U.S. Application Data
(60) Provisional application No. 62/101,586, filed on Jan. 9, 2015.

(51) Int. Cl. A61F 9/00; A61M 31/00; B65D 83/00

(52) U.S. Cl. CPC A61F 9/0026; B65D 83/0055; A61M 31/00; A61M 2210/0662; A61M 2209/08

(57) ABSTRACT

A handling tool adapted to removably receive and handle a bottle of eye drop fluid having an arcuate section and a linear section. The arcuate section is configured with a partial cylinder. The diameter of the partial cylinder adapted to removably receive and handle a bottle. The partial cylinder has spaced parallel opening edges. The linear section is formed of two lineal legs, each leg has an interior end integrally fabricated with the arcuate section at the opening edges and an exterior end. The linear legs are adapted to be squeezed together, compressing the arcuate section and deforming the bottle when inverted to dispense eye drop fluid.

### RELATED APPLICATION

The present patent application claims priority to the corresponding provisional patent application Ser. No. 62/101,586, entitled “FLUID HANDLING AND DISPENSING TOOL” filed. on Jan. 9, 2015.

### FIELD OF THE INVENTION

The present invention relates to a fluid handling and dispensing tool and more particularly pertains to a tool used to aid in the dispensing of drops from small bottles with flexible sides and a dropper top such as those used for dispensing eye and ear drops.

### DESCRIPTION OF THE PRIOR ART

The use of tools to handle and dispense drops from a bottle is known in the prior art. More specifically, tools previously devised and utilized for the purpose of dispensing drops from a bottle are known to consist basically of familiar, expected, and obvious structural configurations, notwithstanding the myriad of designs encompassed by the crowded prior art which has been developed for the fulfillment of countless objectives and requirements.

By way of example, U.S. Pat. No. 3,945,381 to Silver discloses an Eye Drop Dispenser and Cup. U.S. Pat. No. 6,423,040 to Benktzon, Bjarke, Cranfoord, Himbert, Mannberg, Svensson and Tornsten discloses an Eye Fluid Applicator. U.S. Pat. No. 4,134,403 to Johnson, Barta, and Cotner discloses an Eye Drop Dispenser Attachable Positioner. Lastly, U.S. Pat. No. 6,814,265 to Clifford, McCune and Ashlin discloses a Device for Dispensing Fluid Medicine.

While these devices fulfill their respective, particular objectives and requirements, the aforementioned devices do not describe fluid handling and dispensing tool that allows for dispensing fluid from a bottle adapted to dispense drops upon the squeezing and deforming of the side wall of the bottle in an accurate, safe, convenient, and economical manner.

In this respect, the fluid handling and dispensing tool according to the present invention substantially departs from the conventional concepts and designs of the prior art, and in doing so provides an apparatus primarily developed for the purpose of removably receiving and handling a bottle of eye and/or ear drop fluid and for dispensing the eye and/or ear drop fluid from the bottle in an accurate manner, the receiving and handling and dispensing being done in a safe, convenient, and economical manner.

Therefore, it can be appreciated that there exists a continuing need for a new and improved fluid handling and dispensing tool which can be used for the purpose of removably receiving and handling an eye and/or ear drop bottle and to invert and squeeze the side wall of the drop bottle to accurately dispense the drop fluid into an eye or ear of a patient in an accurate, safe, convenient, and economical manner. In this regard, the present invention substantially fulfills this need.

### SUMMARY OF THE INVENTION

In view of the disadvantages inherent in the known types of devices use to aid in the dispensing of drops from a bottle now present in the prior art, the present invention provides an improved fluid handling and dispensing tool. As such, the general purpose of the present invention, which will be described subsequently in greater detail, is to provide anew and improved fluid handling and dispensing tool and method which has all the advantages of the prior art and none of the disadvantages.

To attain this, the present invention essentially comprises an arcuate section and a linear section. The arcuate section is configured with a partial cylinder. The diameter of the partial cylinder is adapted to removably receive and handle a bottle. The partial cylinder has spaced parallel opening edges. The linear section is formed of two lineal legs, each leg has an interior end integrally fabricated with the arcuate section at the opening edges and an exterior end. The linear legs are adapted to be squeezed together, compressing the arcuate section and deforming the bottle when inverted to dispense a drop of fluid. The two linear legs are of sufficient length as to provide leverage when compressing a bottle to dispense drops. The fluid handling and dispensing tool is integrally molded of a semi-rigid material.

A feature of the invention includes radially spaced axial projections forming ribs in the interior surface of the arcuate section. The height of the ribs is such that the inside diameter of the cylinder including the ribs is slightly smaller than the outside diameter of the bottle, providing an interference fit. The interference fit facilitates gripping and prevents rotation of the bottle.

Another feature of the invention are knurled, channeled or otherwise textured portions of the exterior surface adjacent to the exterior ends of the linear legs to facilitate gripping.

It is another feature of the invention to provide a surface adapted to allow for the printing of indicia for advertising and marketing purposes.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims attached.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved fluid handling and dispensing tool which has all of the advantages of the prior art dispensing tools and none of the disadvantages.

It is another object of the present invention to provide a new and improved fluid handling and dispensing tool which may be easily and efficiently manufactured and marketed.

It is further object of the present invention to provide a new and improved fluid handling and dispensing tool which is of durable and reliable constructions.

An even further object of the present invention is to provide a new and improved fluid handling and dispensing tool which is susceptible of a low cost of manufacture with regard to both materials and labor, and which accordingly is then susceptible of low prices of sale to the consuming public, thereby making such fluid handling and dispensing tool economically available to the buying public.

Even still another object of the present invention is to provide a fluid handling and dispensing tool to provide a mechanical aid in dispensing the drop fluid from a bottle in an accurate manner.

Another object of the present invention is to provide an improved means for locating, grasping, picking up and stabilizing the bottle.

Lastly, it is an object of the present invention to provide a new and improved fluid handling and dispensing tool used to provide leverage to accurately control the amount of fluid dispensed by allowing greater control of the pressure placed on the bottle.

These together with other objects of the invention, along with the various features of novelty which characterize the invention, are pointed out with particularity in the claims annexed to and forming a part of this disclosure.

For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is perspective view of a fluid handling and dispensing tool constructed in accordance with the principles of the present invention.

FIG. 2 is a top view of a fluid handling and dispensing tool constructed in accordance with the principles of the present invention.

FIG. 3 is a side view of a fluid handling and dispensing tool constructed in accordance with the principles of the present invention.

The same reference numerals refer to the same parts throughout the various Figures.

### DESCRIPTION OF THE PREFERRED EMBODIMENT

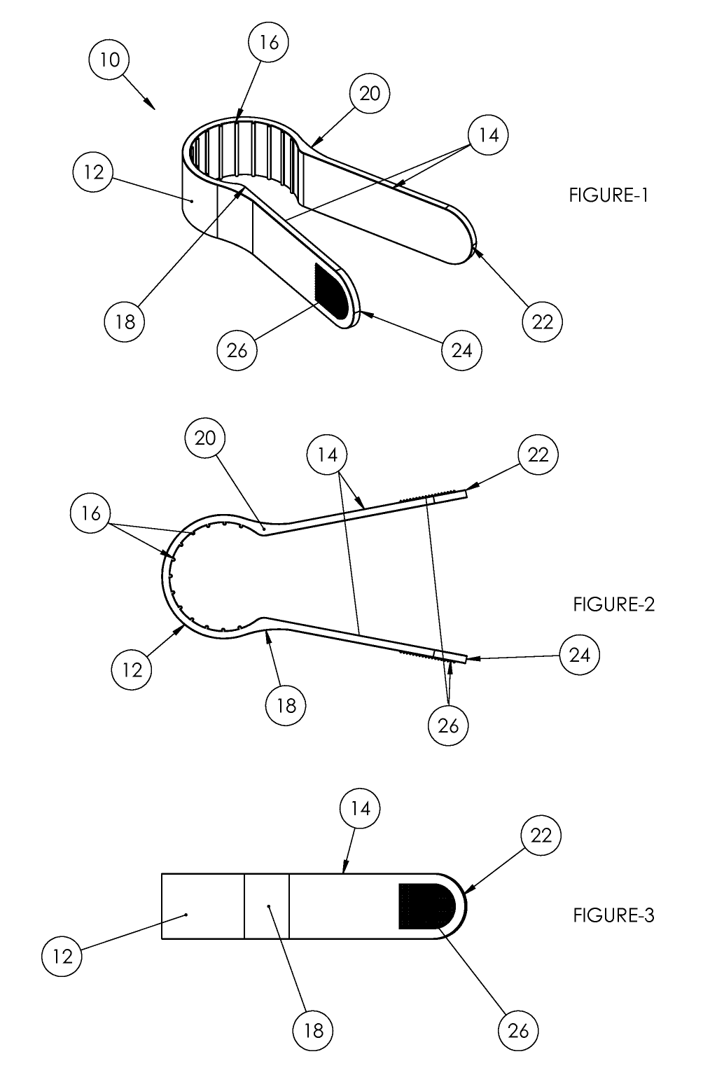

With reference now to the drawings, and in particular to FIG. 1 thereof, the preferred embodiment of the new and improved fluid handling and dispensing tool embodying the principles and concepts of the present invention and generally designated by the reference numeral 10 will be described.

The present invention, the fluid handling and dispensing tool 10 is comprised of a plurality of components. Such components are individually configured and correlated with respect to each other so as to attain the desired objective. In their broadest context such include an arcuate section constructed to receive the body of a bottle fabricated of a flexible plastic and a linear section comprised of two legs. In this broad context, first provided is a handling tool adapted to removably receive and handle a bottle of drop fluid, the handling tool having an arcuate section and a linear section. The arcuate section is configured with a partial cylinder. The diameter of the partial cylinder adapted to removably receive and handle a bottle. The partial cylinder has spaced parallel opening edges. The linear section is formed of two lineal legs, each leg has an interior end integrally fabricated with the arcuate section at the opening edges and an exterior end. The linear legs are adapted to be squeezed together, compressing the arcuate section and deforming the bottle when inverted to dispense eye drop fluid. The handling tool, including the arcuate section and the lineal section, is fabricated of a semi-rigid material chosen from the class of semi-rigid materials including of rubber, polymers and metal. Such components are individually configured and correlated with respect to each other so as to attain the desired objective.

In the preferred embodiment of the fluid handling and dispensing tool, designated by reference numeral 10, first provided is a fluid handling and dispensing tool 10 adapted to removably receive and handle an eye/ear drop bottle and to invert and squeeze the side wall of the eye/ear drop bottle to accurately dispense the drop fluid into an eye/ear of a patient. The handling tool has an arcuate section 12 and a linear section 14. The arcuate section is formed with a configuration of a partial cylinder. The partial cylinder extends for 270 degrees plus or minus 10 percent. The partial cylinder has an interior surface with a diameter of 0.992 inches plus or minus 10 percent. The partial cylinder has spaced parallel opening edges. The interior surface is formed with radial projections 16 formed as ribs crosswise along the interior surface, the ribs forming an interior diameter to facilitate gripping of the bottle during use. The interior diameter formed by the radial projections being slightly smaller than the exterior diameter of the bottle. The linear section 14 is formed of two similarly configured lineal legs, each leg having an interior end 18, 20 integrally fabricated with the arcuate section at the opening edges and an exterior end 22, 24 projecting axially from the partial cylinder. The integrally fabricated portion of the interior ends and the opening edges is reinforced to prevent breakage. Each leg has an exterior surface. A portion of the exterior surface adjacent to the exterior end is knurled, channeled or otherwise textured to facilitate gripping. Each leg is 1.94 inches long plus or minus 10 percent. The handling tool including the arcuate section and the lineal section has a length of 0.63 inches plus or minus 10 percent. The handling tool including the arcuate section and the lineal section is fabricated of a semi rigid material taken from a class of semi-rigid materials including plastic and polymers.

The fluid handling and dispensing tool was developed to aid patients in dispensing eye/ear drops. The tool allows for a drop bottle to be installed into the cylindrical portion of the tool for easy handling and locating. The patient can then squeeze the handles applying pressure to the sides of the bottle and releasing the prescribed amount of drops. The tool provides leverage to accurately control the amount of fluid dispensed by allowing greater control of the pressure placed on the bottle. Patients whose hands have become debilitated due to age, arthritis, or disease will appreciate the mechanical advantage provided by the tool to grip and stabilize the bottle when dispensing drops. The interior ribbing of the cylindrical portion helps secure the bottle versus a smooth inner surface, facilitating gripping and preventing rotation of the bottle. The bottle can remain installed in the tool until empty providing an improved means for locating, grasping, and picking up the bottle.

As to the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed as being new and desired to be protected by Letters Patent of the United States is as follows:

1. A fluid handling and dispensing system comprising:
a handling tool adapted to removably receive and handle a bottle containing fluid having flexible sides formed in a cylindrical configuration and a dropper tip, the handling tool having an arcuate section and a linear section;
the arcuate section having a configuration of a partial cylinder, the partial cylinder having an interior surface with a diameter adapted to removably receive and handle the bottle, the partial cylinder having spaced parallel opening edges; and
the linear section being formed of two lineal legs, each leg having an interior end integrally fabricated with the arcuate section at the opening edges, each leg having an exterior end, each leg having an exterior surface, the linear legs adapted to be squeezed together to deform the bottle when inverted to dispense drops of fluid, the handling tool including the arcuate section and the lineal section being fabricated of a semi-rigid material chosen from the class of semi-rigid materials including rubber, polymers and metal.

2. The fluid handling and dispensing system of claim 1 wherein the interior surface is formed with axial projections to facilitate gripping of the bottle during use.

3. The fluid handling and dispensing system of claim. 2 wherein the axial projections are formed as ribs crosswise along the interior surface.

4. The fluid handling and dispensing system of claim 3 wherein the axial projections are spaced at 20 degree intervals along the interior diameter of the arcuate section.

5. The fluid handling and dispensing system of claim 1 wherein, a portion of the exterior surface adjacent to the exterior end is knurled, channeled or otherwise textured to facilitate gripping.

6. A fluid handling and dispensing system, the system comprising, in combination:
a handling tool adapted to removably receive and handle a bottle and to invert and squeeze the side wall of the bottle to accurately dispense the eye/ear drop fluid into an eye/ear of a patient, the handling tool having an arcuate section and a linear section, the arcuate section having a configuration of a partial cylinder, the partial cylinder extending for 270 degrees plus or minus 10 percent, the partial cylinder having an interior surface with a diameter of 0.992 inches plus or minus 10 percent, the partial cylinder having spaced parallel opening edges, the interior surface being formed with axial projections to facilitate gripping of the bottle during use, the axial projections forming an interior diameter, the interior diameter formed by the axial projections being slight smaller than the exterior diameter of the bottle, the linear section being formed of two similarly configured lineal legs, each leg having an interior end integrally fabricated with the arcuate section at the opening edges, each leg having an exterior end in a semi-circular configuration, each leg having an exterior surface, a portion of the exterior surface adjacent to the exterior end being knurled, channeled or otherwise textured to facilitate gripping, each leg being 1.94 inches long plus or minus 10 percent, the handling tool including the arcuate section and the lineal section having a length of 0.63 inches plus or minus 10 percent, the handling tool including the arcuate section and the lineal section being fabricated of a semi rigid material taken from a class including plastic and polymers.
